SEQUENCE LISTING (17 sequences)

SEQ ID NO: 1 - protein, 5 aa, Rattus norvegicus
NYYMA

SEQ ID NO: 2 - protein, 17 aa, Rattus norvegicus
YISPGGGSTYYRDSVKG

SEQ ID NO: 3 - protein, 9 aa, Rattus norvegicus
DRENWELDY

SEQ ID NO: 4 - protein, 11 aa, Rattus norvegicus
QASQNINKYIA

SEQ ID NO: 5 - protein, 7 aa, Rattus norvegicus
YTSSLES

SEQ ID NO: 6 - protein, 8 aa, Rattus norvegicus
LQYDNLRT

SEQ ID NO: 7 - protein, 137 aa, Rattus norvegicus
MDIRLSLVFLVLFIKGVQCEVQLVESGGGLVQPGRSLKLSCAASGFTFSNYYMAWVRQAPTKGLEWVAYISPGGGSTYYRDSVKGRFTISRDNAKSTLYLQMDSLRSEDTATYYCTTDRENWELDYWGQGVMVTVSS

SEQ ID NO: 8 - protein, 126 aa, Rattus norvegicus
MRTSIQLLGLLLFWLHDAQCDIQMTQSPPSLSASLGDKVTITCQASQNINKYIAWYQQKPGKAPRLLIHYTSSLESGTPSRFSGSGSGRDYSFSISNVESEDIASYYCLQYDNLRTFGGGTKLELK

SEQ ID NO: 9 - protein, 5 aa, Mus musculus
DYYIN

SEQ ID NO: 10 - protein, 17 aa, Mus musculus
RIYPLTYNTEYNEKFKG

SEQ ID NO: 11 - protein, 11 aa, Mus musculus
SFGYDVKFMDY

SEQ ID NO: 12 - protein, 11 aa, Mus musculus
KASQSVSNDVT

SEQ ID NO: 13 - protein, 7 aa, Mus musculus
YASTRYT

SEQ ID NO: 14 - protein, 9 aa, Mus musculus
QQDYSSPWT

SEQ ID NO: 15 - protein, 139 aa, Mus musculus
MGWSWIFLFLLSGTAGVHCQIQLQQSGPELVKPGASVKISCKASDYTFTDYYINWVKQKPGQGLEWIGRIYPLTYNTEYNEKFKGKATLTVDTSSSTAYMQLISLTSEDTAVYFCARSFGYDVKFMDYWGQGTSVTVSS

SEQ ID NO: 16 - protein, 127 aa, Mus musculus
MKSQTQVFVFLLLCVSGAHGSIVMTQTPKFLLVSAGDRITITCKASQSVSNDVTWYQQKPGQSPKLLIYYASTRYTGVPDRFTGSGYGTDFTFTINTVQAEDLAVYFCQQDYSSPWTFGGGTQLEIK

SEQ ID NO: 17 - protein, 1049 aa, Homo sapiens [sequence omitted]

(19) United States
(12) Patent Application Publication
(10) Pub. No.: US 2021/0199667 A1
(43) Pub. Date: Jul. 1, 2021

(54) ANALYSIS OF SOLUBLE TLR7 IN HUMAN-DERIVED SAMPLE

(71) Applicant: THE UNIVERSITY OF TOKYO, Tokyo (JP)

(72) Inventors: Kensuke MIYAKE, Tokyo (JP); Yusuke MURAKAMI, Tokyo (JP); Yuji MOTOI, Tokyo (JP); Toshiyuki SHIMIZU, Tokyo (JP); Umeharu OHTO, Tokyo (JP)

(73) Assignee: THE UNIVERSITY OF TOKYO, Tokyo (JP)

(21) Appl. No.: 17/058,489

(22) PCT Filed: May 30, 2019
(86) PCT No.: PCT/JP2019/021490

§ 371 Date: Nov. 24, 2020

(30) Foreign Application Priority Data
May 31, 2018 (JP) 2018-104755

(51) Int. Cl. G01N 33/68; C07K 16/28

(52) U.S. Cl. CPC G01N 33/6854; C07K 2317/76; G01N 2800/24; C07K 16/2896

(57) ABSTRACT

An object of the present invention is to provide a technique for detecting human TLR7. Human TLR7 in a sample can be detected through an ELISA method or the like using an anti-human TLR7 antibody. The present invention enables use of human TLR7 as a marker indicating the pathological condition of an autoimmune disease.

### TECHNICAL FIELD

The present invention relates to a technique for detecting toll-like receptor 7 (TLR7). In particular, the present invention relates to detection of soluble TLR7, and also to screening for substances targeting TLR7, treatment targeting TLR7, and so on.

### BACKGROUND ART

Toll-like receptors (TLRs), forming a family of pathogen sensors, induce an activation signal in response to a pathogen component, and induce phylactic reaction. TLRs are not only important in phylaxis, but also involved in induction of inflammation in pathological condition of autoimmune diseases and so on. For example, NPL 1 proposes use of TLRs in serum as an index of radiation pneumonitis in patients with non-small cell lung cancer (NSCLC).

Among about 10 TLRs, TLR3, TLR7, TLR8, and TLR9 are distributed in endoplasmic reticulum, an intracellular organelle, and recognize a nucleic acid derived from bacteria and viruses. TLR7 and TLR8 recognize single-stranded RNA, and TLR9 recognizes non-methylated single-stranded DNA including a CpG motif (CpG-DNA).

In contrast to double-stranded RNA unique to viruses, however, single-stranded RNA and DNA are not very different from nucleic acids derived from a host, and evoke auto-aggressive reaction, eventually causing an autoimmune disease, if the ligand recognition mechanism by TLRs is not strictly controlled.

In this regard, the autoimmune reaction by TLR7 is regulated through limiting the site that recognizes a nucleic acid to the endolysosome (NPL 2). In steady state, autologous nucleic acids extracellularly present is quickly degraded, and hence do not reach the intracellular endolysosome, and thus is not recognized by TLR7. By contrast, microbial nucleic acids are protected by cell walls of bacteria or viral particles, and hence allowed to reach the endolysosome, released on the arrival there and recognized by TLR7.

If an autologous nucleic acid comes to have resistance to degradation and becomes able to reach the endolysosome through interaction with an anti-microbial peptide or autoantibody, on the other hand, TLR7-dependent autoimmune reaction is caused. Actually, it has been suggested that TLR7 has relation to psoriasis and systemic lupus erythematosus (SLE) (NPTLs 3 to 5).

Therefore, TLR7 is thought to be a therapeutic target for TLR7-dependent autoimmune diseases such as psoriasis and SLE, and various methods have been previously proposed to suppress TLR7 expression and functions. Specifically, a method using oligo DNA having antagonistic effect to TLR7, a method using microRNA that suppresses TLR7 expression, and so on have been attempted. However, safety of nucleic acid drugs is generally unknown, and there is still the possibility that complete suppression of TLR7 functions causes danger such as infections.

TLR7 has been believed to be localized in the endolysosome to limit autoimmune reaction and completely separated from cell surfaces, and hence some researchers have thought that antibodies that act only on cell surfaces are not applicable; however, an antibody drug said to be superior in safety and specificity has been proposed (PTL 1).

### CITATION LIST

### Patent Literature

PTL 1: International Publication No. WO 2014/174704

### Non Patent Literature

NPL 1: Int. J. Clin. Exp. Pathol., 711:8087-8095 (2014)
NPL 2: Barton, G. et al. d Medzhitov, R. Nat Immunol 7, pp. 49-56 (2006)
NPL 3: Lande, R. et al. Nature 449, pp. 564-569 (2007)
NPL 4: Christensen, S. R. et al. Immunity 25, pp. 417-428 (2006)
NPL 5: Ehlers, M. et al. J Exp Med 203, pp. 553-561 (2006)

### SUMMARY OF INVENTION

### Technical Problem

In systemic lupus erythematosus (SLE) and related autoimmune diseases, inflammation due to autoimmune response is induced and said to be largely involved in the pathological condition; however, much remains unclear on causes of these diseases. Steroids having nonspecific anti-inflammatory effect have been used as therapeutic drugs, but their side effects cause significant problems for some cases, and hence it is critical to identify a therapeutic target and develop a more specific therapeutic method.

TLR7 is an RNA sensor localized in endosomes and lysosomes, and it has been suggested that TLR7 is involved in pathological condition of autoimmune diseases, in particular, in the autoimmune disease SLE. Findings on TLR7 are, however, primarily those with model mice, and results indicating the participation of TLR7 with human samples have not been sufficient.

Studies on SLE model mice thus suggested that TLR7 is involved in the pathological condition, and hence TLR7 is expected to be a therapeutic target molecule for connective tissue diseases and so on including SLE; nevertheless, no technique for detecting TLR7 in a simple manner has been established, and a new procedure that enables examination of the participation of TLR7 in the pathological condition has been required. In particular, procedures applicable to analysis to examine whether TLR7 is involved in autoimmune diseases such as SLE are limited in humans as compared with mice, and it has been difficult to analyze the relation between TLR7 and the pathological condition.

### Solution to Problem

The present inventors diligently examined the above problems, and succeeded in actually obtaining a plurality of monoclonal antibodies against human TLR7, and establishing an analysis method capable of detecting TLR7, and on the basis of these eventually completed the present invention.

The present invention includes, but is not limited to, the followings.

(1) A method for analyzing a human-derived body fluid sample, comprising:

detecting human TLR7 in a sample from a test subject by using an anti-human TLR7 antibody; and

comparing a detection result on the sample from the test subject with a detection result on a sample derived from a healthy individual.

(2) The method according to (1), wherein the human TLR7 is detected through enzyme immunoassay and/or immunoprecipitation.
(3) The method according to (1) or (2), wherein the human TLR7 in each of the samples is detected by using a plurality of anti-human TLR7 antibodies.
(4) The method according to any one of (1) to (3), wherein each of the samples is a sample derived from human body fluid.
(5) The method according to any one of (1) to (4), wherein each of the samples comprises human plasma and/or serum.
(6) The method according to any one of (1) to (5), wherein the test subject is a patient having an autoimmune disease or an individual suspected to have an autoimmune disease.
(7) A method for evaluating effect of a medicine targeting human TLR7 in a human, comprising detecting human TLR7 in a sample derived from a human by using an anti-human TLR7 antibody.
(8) The method according to (7), wherein the medicine targeting human TLR7 is a medicine for an autoimmune disease.
(9) The method according to (7) or (8), wherein the medicine targeting human TLR7 is a medicine for SLE.
(10) An agent for examining an autoimmune disease, comprising an anti-human TLR7 antibody.
(11) The agent according to (10), for analyzing a human-derived body fluid sample.
(12) The agent according to (10) or (11), wherein the autoimmune disease is SLE.

### Advantageous Effects of Invention

The present invention enables detection of TLR7 in a simple manner. In particular, the present invention enables detection of soluble TLR7, and is applicable even to screening for substances targeting TLR7, treatment targeting TLR7, and so on.

The present invention can measure soluble TLR7 in body fluid such as human serum, and hence facilitates examination of the relation between the variation of soluble TLR7 and pathological condition in autoimmune diseases. In conventionally known analyses with human samples, examination has been made on TLR7 expression and response to a TLR7 ligand with use of leukocytes derived from peripheral blood; however, these analyses need fresh blood, and it is by no means easy to obtain samples. Since the present invention enables analysis of more samples by using body fluid, the present invention is extremely useful for analysis, treatment, and research of diseases and pathological condition associated with TLR7.

### BRIEF DESCRIPTION OF DRAWINGS

FIG. 1 shows results of analysis of gene sequences in Experiment 1 (rE3 and LTM3, each underline indicates CDR).

FIG. 2 shows histograms representing results in Experiment 2.

FIG. 3 shows graphs demonstrating that rE3 and LTM3 differ in their epitopes.

FIG. 4 shows a standard curve for monkey TLR7 prepared in Experiment 3.

FIG. 5 shows results of detecting soluble TLR7 through immunoprecipitation and a Western blotting method in Experiment 4.

FIG. 6 shows a graph representing results in Experiment 5 (−: mean value, *: p<0.05; ***: p<0.001).

### DESCRIPTION OF EMBODIMENTS

In the present invention, an antibody against human TLR7 (herein, also referred to as hTLR7) is used. The anti-human TLR7 antibody according to the present invention is not limited and may be any anti-human TLR7 antibody that recognizes the extracellular domain of human TLR7, and may be not only the whole of such an antibody but also a functional fragment thereof.

Known as the TLR family are 12 types for mice and 10 types for humans. TLR1, TLR2, TLR4, TLR5, and TLR6 are distributed on cell surfaces, and recognize lipoprotein, glycolipid such as LPS, and protein such as flagellin, which are bacterial membrane components. TLR3, TLR7, TLR8, and TLR9 are distributed in endoplasmic reticulum, an intracellular organelle, and recognize nucleic acids derived from bacteria and viruses.

TLRs are type I membrane proteins, and extracellularly include an LRR (Leucine rich repeat). On recognizing a pathogen component, TLRs perform signal transduction with an intracellular TIR (Toll/IL-1R homology) domain. A TLR that has recognized a ligand intracellularly transduces a signal via the TIR domain, thereby, eventually activating transcription factors such as NF-κB and an IRF (Interferon-Regulatory Factor) family and inducing production of inflammatory cytokines (such as IL-6, IL-12, and TNFα), inflammatory chemokines (such as RANTES), and type I interferons (IFNα and IFNβ) to locally cause an appropriate innate immune response. Such immune response via TLRs is essential for host defense, and it has been reported that deficiency of a molecule associated with the TLR response leads to susceptibility to infection by various pathogens. However, it has been pointed out that once an autologous substance becomes an endogenous ligand to a TLR for some reasons, the autologous substance may cause chronic inflammation.

TLR7 is known to be actually involved in the onset of SLE and psoriasis. Anti-TLR7 antibodies bind to cell surface TLR7 and inhibit the TLR7 response of the cell, thereby preventing abnormal immune activation to contribute to treatment or prevention of diseases.

The nucleotide sequence of cDNA for human TLR7 is registered, for example, in GenBank as Accession No.: NM 016562, and variants thereof are also known. The amino acid sequence of human TLR7 (variant 2) is set forth in SEQ ID NO: 17. The anti-TLR7 antibody according to the present invention binds to soluble TLR7. Soluble TLR7 is a protein at least including the extracellular domain of TLR7.

One embodiment of the anti-TLR7 antibody to be used in the present invention includes at least one of the following CDRs:

(a) a heavy chain CDR1 consisting of an amino acid sequence represented by SEQ ID NO: 1;
(b) a heavy chain CDR2 consisting of an amino acid sequence represented by SEQ ID NO: 2;
(c) a heavy chain CDR3 consisting of an amino acid sequence represented by SEQ ID NO: 3;
(d) a light chain CDR1 consisting of an amino acid sequence represented by SEQ ID NO: 4;
(e) a light chain CDR2 consisting of an amino acid sequence represented by SEQ ID NO: 5; and
(f) a light chain CDR3 consisting of an amino acid sequence represented by SEQ ID NO: 6.

Another embodiment of the anti-TLR7 antibody to be used in the present invention includes at least one of the following CDRs:

(g) a heavy chain CDR1 consisting of an amino acid sequence represented by SEQ ID NO: 9;
(h) a heavy chain CDR2 consisting of an amino acid sequence represented by SEQ ID NO: 10;
(i) a heavy chain CDR3 consisting of an amino acid sequence represented by SEQ ID NO: 11;
(j) a light chain CDR1 consisting of an amino acid sequence represented by SEQ ID NO: 12;
(k) a light chain CDR2 consisting of an amino acid sequence represented by SEQ ID NO: 13; and
(l) a light chain CDR3 consisting of an amino acid sequence represented by SEQ ID NO: 14.

The anti-TLR7 antibody to be used in the present invention may be an antibody including at least two, at least three, at least four, at least five, or all of heavy chain CDR1 to CDR3 and light chain CDR1 to CDR3 of (a) to (f). The anti-TLR7 antibody to be used in the present invention may be an antibody including at least two, at least three, at least four, at least five, or all of heavy chain CDR1 to CDR3 and light chain CDR1 to CDR3 of (g) to (1).

The anti-TLR7 antibody to be used in the present invention may be an antibody in which at least one of heavy chain CDR1 to CDR3 and light chain CDR1 to CDR3 of (a) to (f) includes deletion, substitution, or addition of one or two amino acids in the amino acid sequence. The anti-TLR7 antibody to be used in the present invention may be an antibody in which at least one of heavy chain CDR1 to CDR3 and light chain CDR1 to CDR3 of (g) to (1) includes deletion, substitution, or addition of one or two amino acids in the amino acid sequence.

Herein, the term “amino acid” is used in the broadest sense, and encompasses not only natural amino acids but also non-natural amino acids such as amino acid variants and derivatives. Examples of amino acids include, but are not limited to, natural protein-constituting L-amino acids; D-amino acids; chemically modified amino acids such as amino acid variants and derivatives; natural non-protein-constituting amino acids such as norleucine, β-alanine, and ornithine; and chemically synthesized compounds having properties known in the art as characteristics of amino acids. Examples of non-natural amino acids include, but are not limited to, α-methylamino acids (such as α-methylalanine), D-amino acids, histidine-like amino acids (such as 2-amino-histidine, β-hydroxy-histidine, homohistidine, α-fluoromethyl-histidine, and α-methyl-histidine), amino acids with excessive methylene in the side chain (“homo” amino acids), and amino acids in which a carboxylic acid functional group amino acid in the side chain is substituted with a sulfonic acid group (such as cysteic acid).

When the phrase “including deletion, substitution, or addition of one or two amino acids” is used herein, the number of amino acids for deletion, substitution, and so on is not limited as long as the resulting CDR set retains the antigen recognition function. The position of deletion, substitution, or addition in each CDR may be the N terminus, the C terminus, or any intermediate position, as long as the resulting CDR set retains the antigen recognition function.

The anti-TLR7 antibody to be used in the present invention may be an anti-TLR7 antibody in which at least one of heavy chain CDR1 to CDR3 and light chain CDR1 to CDR3 of (a) to (f) consists of an amino acid sequence having an identity of 90% or higher, 95% or higher, or 98% or higher to the corresponding amino acid sequence(s) of those represented by SEQ ID NOs: 1 to 6, as long as the resulting CDR set retains the function of CDRs of an anti-TLR7 antibody. The anti-TLR7 antibody to be used in the present invention may be an anti-TLR7 antibody in which at least one of heavy chain CDR1 to CDR3 and light chain CDR1 to CDR3 of (g) to (1) consists of an amino acid sequence having an identity of 90% or higher, 95% or higher, or 98% or higher to the corresponding amino acid sequence(s) of those represented by SEQ ID NOs: 9 to 14, as long as the resulting CDR set retains the function of CDRs of an anti-TLR7 antibody.

Herein, the phrase “having an identity of Y % or higher to an amino acid sequence represented by SEQ ID NO: X” refers to the situation that when two polypeptides are aligned so as to maximize the concordance of the amino acid sequences, the fraction of the number of matching amino acid residues to the total number of amino acids represented by SEQ ID NO: X is Y % or higher.

The anti-TLR7 antibody to be used in the present invention may be any of the followings:

(1) an antibody including a heavy chain variable region including an amino acid sequence represented by SEQ ID NO: 7 and a light chain variable region including an amino acid sequence represented by SEQ ID NO: 8;
(2) an antibody including a heavy chain variable region and/or light chain variable region including an amino acid sequence(s) formed by deleting, substituting, or adding one or several amino acids in an amino acid sequence(s) represented by SEQ ID NO(s): 7 and/or 8;
(3) an antibody including a heavy chain variable region and/or light chain variable region including an amino acid sequence(s) having an identity of 70% or higher to an amino acid sequence(s) represented by SEQ ID NO(s): 7 and/or 8; and
(4) an antibody that recognizes an epitope for any antibody of (1) to (3).

In another embodiment, the anti-TLR7 antibody to be used in the present invention may be any of the followings:

(1) an antibody including a heavy chain variable region including an amino acid sequence represented by SEQ ID NO: 15 and a light chain variable region including an amino acid sequence represented by SEQ ID NO: 16;
(2) an antibody including a heavy chain variable region and/or light chain variable region including an amino acid sequence(s) formed by deleting, substituting, or adding one or several amino acids in an amino acid sequence(s) represented by SEQ ID NO(s): 15 and/or 16;
(3) an antibody including a heavy chain variable region and/or light chain variable region including an amino acid sequence(s) having an identity of 70% or higher to an amino acid sequence(s) represented by SEQ ID NO(s): 15 and/or 16; and
(4) an antibody that recognizes an epitope for any antibody of (1) to (3).

Herein, the phrase “an antibody including a heavy chain variable region and/or light chain variable region including an amino acid sequence(s) formed by deleting, substituting, or adding one or several amino acids in an amino acid sequence(s) represented by SEQ ID NO(s): A and/or B” refers to the situation that the heavy chain variable region consists of a sequence formed by deleting, substituting, or adding one or several amino acids in the amino acid sequence represented by SEQ ID NO: A, and/or the light chain variable region consists of a sequence formed by deleting, substituting, or adding one or several amino acids in the amino acid sequence represented by SEQ ID NO: B. The number of deletion, substitution, or addition is not limited as long as the antibody including the resulting heavy chain variable region and light chain variable region specifically binds to the antigen, and can be 1, 2, 3, 4, 5, 6, 7, 8, 9, or 10. The position of deletion, substitution, or addition is not limited, similarly, as long as the antibody including the resulting heavy chain variable region and light chain variable region specifically binds to the antigen.

Herein, the phrase “an antibody including a heavy chain variable region and/or light chain variable region including an amino acid sequence(s) having an identity of 70% or higher to an amino acid sequence(s) represented by SEQ ID NO(s): A and/or B” refers to the situation that the heavy chain variable region has an identity of 70% or higher to the amino acid sequence represented by SEQ ID NO: A, and/or the light chain variable region has an identity of 70% or higher to the amino acid sequence represented by SEQ ID NO: B. Each identity is not limited as long as the antibody including the resulting heavy chain variable region and light chain variable region specifically binds to TLR7, and can be 80% or higher, 85% or higher, 90% or higher, 95% or higher, or 98% or higher.

The anti-TLR7 antibody to be used in the present invention may be an anti-TLR7 antibody that binds within the region from position 276 to position 313 of the amino acid sequence of human TLR7 represented by SEQ ID NO: 17.

The anti-TLR7 antibody to be used in the present invention may be a monoclonal antibody or a polyclonal antibody. The anti-TLR7 antibody to be used in the present invention may be in any isotype of IgG, IgM, IgA, IgD, and IgE.

The anti-TLR7 antibody to be used in the present invention may be, but is not limited to, any mouse antibody, human-type CDR-transplanted antibody, human-type chimeric antibody, humanized antibody, or full human antibody, or low-molecular-weight antibody that binds to cell surface TLR7.

Human-type CDR-transplanted antibodies are antibodies obtained by substituting CDRs of an antibody of an animal other than humans with CDRs of a human antibody.

Human-type chimeric antibodies are antibodies consisting of a variable region derived from an antibody of an animal other than humans and a constant region derived from a human antibody. Humanized antibodies are antibodies obtained by incorporating a part derived from an antibody of a human into an antibody of an animal other than humans with some highly safe regions remained, and the concept encompasses human-type chimeric antibodies and human-type CDR-transplanted antibodies.

Herein, a “low-molecular-weight antibody” refers to an antibody fragment or a product obtained by binding an arbitrary molecule to an antibody fragment, wherein the antibody fragment or product recognizes the epitope for the original antibody. Specific examples thereof include, but are not limited to, Fab, which consists of VL, VH, CL, and CHI regions; F(ab′)2, in which two Fab molecules are linked via disulfide bonds in the hinge region; Fv, which consists of VL and VH; scFV, which is a single-chain antibody in which VL and VH are linked together via an artificial polypeptide linker; and sdFv, Diabodies, and sc(Fv)₂.

In producing the anti-TLR7 antibody to be used in the present invention, the production method is not limited, and an example thereof for a monoclonal antibody is such that antibody-producing cells are isolated from a non-human mammal immunized with TLR7 or a fragment thereof, and fused with myeloma cells or the like to produce a hybridoma, and an antibody produced by this hybridoma is purified to obtain a monoclonal antibody. rE3 and LTM3 described later in Examples are monoclonal antibodies produced in such a manner. A polyclonal antibody can be obtained from serum of an animal immunized with TLR7 or a fragment thereof. The fragment of TLR7 for immunization is not limited as long as the resulting antibody binds to cell surface TLR7 and inhibits the function, and examples of such fragments include a TLR7 fragment including an amino acid sequence from position 276 to position 313 of SEQ ID NO: 17.

In the case that an anti-TLR7 antibody having a specific amino acid sequence is produced, for example, the anti-TLR7 antibody can be produced in such a manner that an appropriate host is transformed with an expression vector including a nucleic acid encoding an anti-TLR7 antibody, and this transformant is cultured under appropriate conditions to express the antibody, which is isolated/purified in accordance with a known method. Examples of isolation/purification methods include affinity columns using protein A or the like, other chromatography columns, filters, ultrafiltration, salting-out, and dialysis, and these can be appropriately combined.

“Antibody Y that specifically binds to an epitope for antibody X” can be produced after determining the sequence of the epitope as follows.

The epitope on the antigen protein can be determined, for example, in such a manner that many peptides having random sequences are immobilized to a solid phase carrier to form an array, which is reacted with antibody X, and binding is detected with an enzyme-labeled secondary antibody to examine the amino acid sequence of a peptide to which antibody X specifically binds, and homology between this amino acid sequence and the amino acid sequence of the antigen protein is searched. A group of partial peptides of the antigen protein may be provided in advance as peptides to be immobilized to a solid phase carrier.

Alternatively, the epitope on the antigen protein can be determined in such a manner that binding between antibody X and the antigen protein is detected through an ELISA method in the presence of a variety of partial peptides of the antigen protein, and the presence or absence of competitive activity is determined.

Once the sequence of the epitope is successfully determined, those skilled in the art can produce antibody Y which specifically binds to this in accordance with a known method. For example, an antibody which specifically binds to the epitope can be obtained by immobilizing a peptide including the epitope sequence to a solid phase carrier and detecting binding between the peptide and a variety of antibodies.

Here, for “a variety of antibodies” those obtained through immunization of an animal with the antigen protein or a partial peptide thereof may be used, and an antibody library or antibody fragment library produced with a phage display method may be used. In the case that a library produced with a phage display method is used, antibody Y that specifically binds to the epitope can be obtained by immobilizing a peptide including the epitope sequence to a solid phase carrier and repeating panning.

Human chimeric antibodies and human CDR-transplanted antibodies can be produced by cloning an antibody gene from mRNA of a hybridoma that produces an antibody of an animal other than humans and linking the cloned antibody gene to a part of a human antibody gene with a gene recombinant technique.

In the case of human-type chimeric antibodies, a cDNA is synthesized from mRNA of a hybridoma that produces a mouse antibody with reverse transcriptase, and the heavy chain variable region (VH) and light chain variable region (VL) are cloned through PCR, and the sequences are analyzed. Subsequently, 5′ primer including a leader sequence is produced from an antibody nucleotide sequence with a high concordance rate, and a region from a signal sequence to the 3′ end of a variable region is cloned from the cDNA through PCR with the 5′ primer and a variable region 3′ primer. Separately, the heavy chain and light chain constant regions of human IgG1 are cloned, and for the heavy chain and light chain a variable region derived from the mouse antibody and a constant region derived from the human antibody are linked together through an Overlapping Hanging method with PCR, and amplified. The resulting DNA is inserted into an appropriate vector to transform it, and thus a human-type chimeric antibody is successfully obtained.

In the case of CDR-transplanted antibodies, a human antibody variable region with the highest homology to a mouse antibody variable region to be used is selected and cloned, and the CDR nucleotide sequence is modified through site-specific mutation with a megaprimer method. If antigen-specific binding fails through humanization of the amino acid sequence constituting the framework region, some amino acids in the framework may be changed from human type to rat type.

“CDR consisting of an amino acid sequence formed by deleting, substituting, or adding one or two amino acids in an amino acid sequence represented by SEQ ID NO: X” and “CDR consisting of an amino acid sequence having an identity of Y % or higher to an amino acid sequence represented by SEQ ID NO: X” can be produced by using a known method such as a site-specific mutation method, a random mutation method, a chain shuffling method, and a CDR walking method.

It is well-known by those skilled in the art that CDR with more matured affinity can be obtained with any of those methods through displaying antibodies or antibody fragments with a variety of CDR mutations on phage surfaces with a phage display method and screening by using the antigen (e.g., Wu et al., PNAS, 95:6037-6042 (1998); Schier, R. et al., J. Mol. Bio. 263:551-567 (1996); Schier, R. et al., J. Mol. Biol. 255:28-43 (1996); Yang, W. P. et al., J. Mol. Biol., 254:392-403 (1995)). The present invention includes antibodies including CDR matured with such a method.

Other methods for manufacturing antibodies include an Adlib method to obtain an antibody-producing strain from trichostatin A-treated chicken B cell-derived DT40 cell lines (Seo, H. et al., Nat. Biotechnol., 6:731-736, 2002) and a method of producing a human antibody by immunizing a KM mouse, a mouse obtained by breaking a mouse antibody gene and introducing a human antibody gene (Itoh, K. et al., Jpn. J. Cancer Res., 92:1313-1321, 2001; Koide, A. et al., J. Mol. Biol., 284:1141-1151, 1998), and these can be applied to production of the antibody according to the present invention.

In the case that the anti-TLR7 antibody of the present invention is a low-molecular-weight antibody, the low-molecular-weight antibody may be expressed through any of the above methods with a DNA encoding the low-molecular-weight antibody, and may be produced by treating the full-length antibody with an enzyme such as papain and pepsin.

The antibody to be used in the present invention may vary in the amino acid sequence, molecular weight, isoelectric point, presence or absence of a glycan, form, and so on among production methods and purification methods therefor. However, if an antibody obtained has the equivalent function to the antibody of the present invention, the antibody obtained is included in the present invention. If the antibody of the present invention is expressed in prokaryotic cells such as Escherichia coli, for example, a methionine residue is added to the N terminus of the amino acid sequence of the original antibody. The present invention also includes such antibodies.

In the present invention, an antibody may be prepared in accordance with a known method. For an antibody against human TLR7, for example, a non-human animal is immunized with the antigen of interest, and cells derived from the lymph fluid, lymphatic tissue, blood cell sample, or bone marrow are collected from the animal after achievement of immunization, and a hybridoma is established by fusing antibody-producing cells that produce an antibody against the antigen and myeloma cells in accordance with a known method (e.g., Kohler and Milstein, Nature (1975) 256, p. 495-497, Kennet, R, ed., Monoclonal Antibodies, p. 365-367, Plenum Press, N.Y. (1980)), and thus a monoclonal antibody is successfully obtained. Specific examples of such methods are described in WO 2009/48072 (published on Apr. 16, 2009) and WO 2010/117011 (published on Oct. 14, 2010).

The scope of the antibody of the present invention includes, in addition to monoclonal antibodies, gene recombinant antibodies artificially modified, for example, for the purpose of lowering xenoantigenicity to humans, such as chimeric antibodies, humanized antibodies, and human antibodies. These antibodies can be manufactured by using a known method.

Examples of chimeric antibodies can include antibodies whose variable region and constant region are heterologous to each other, such as chimeric antibodies obtained by bonding a variable region of a mouse- or rat-derived antibody to a human-derived constant region (see Proc. Natl. Acad. Sci. U.S.A., 81, 6851-6855 (1984)).

Examples of humanized antibodies can include antibodies obtained by incorporating only CDRs into a human-derived antibody (see Nature (1986) 321, p. 522-525) and antibodies obtained by transplanting not only a CDR sequence but also amino acid residues in a part of a framework into a human antibody by using a CDR transplantation method (International Publication No. WO 90/07861).

In a preferred embodiment, the antibody of the present invention is, for example, a human antibody. For example, an anti-TLR7 human antibody refers to a human antibody having only the gene sequence of an antibody derived from a human chromosome. Such an anti-TLR7 human antibody can be obtained with a method using a human antibody-producing mouse having a human chromosome fragment including a heavy chain and light chain genes of a human antibody. For this method, for example, the followings can be referred to.
Tomizuka, K. et al., Nature Genetics (1997) 16, p. 133-143
Kuroiwa, Y. et. al., Nucl. Acids Res. (1998) 26, p. 3447-3448
Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999
Tomizuka, K. et. al., Proc. Natl. Acad. Sci. USA (2000) 97, p. 722-727

Specifically, such a human antibody-producing mouse is obtained through production of a knockout animal and a transgenic animal followed by mating of the animals to generate such a gene recombinant animal that the loci for the endogenous immunoglobulin heavy chain and light chain have been broken and instead the loci for a human immunoglobulin heavy chain and light chain have been introduced via a vector such as a human artificial chromosome (HAC) vector or a mouse artificial chromosome (MAC) vector.

Also acceptable is the following manner: eukaryotic cells are transformed with cDNAs encoding the heavy chain and light chain of such a human antibody, preferably, with a vector including the cDNAs through a gene recombinant technique, and the transformed cells that produce the gene recombinant human monoclonal antibody are cultured to obtain the antibody from the culture supernatant. For the host here, for example, eukaryotic cells, preferably mammalian cells such as CHO cells, lymphocytes, and myeloma can be used.

Alternatively, anti-TLR7 human antibodies can be obtained with a method of obtaining a human antibody derived from phage display selected from a human antibody library. For this method, for example, the following documents can be referred to.
Wormstone, I. M. et. al, Investigative Ophthalmology & Visual Science. (2002) 43(7), p. 2301-2308
Carmen, S. et. al., Briefings in Functional Genomics and Proteomics (2002), 1(2), p. 189-203
Siriwardena, D. et. al., Ophthalmology (2002) 109(3), 427-431

Those skilled in the art can appropriately determine whether an anti-TLR7 antibody is applicable to the present invention. For example, by confirming at least one of the followings, an anti-TLR7 antibody applicable to detection of human TLR7 can be selected from antibodies obtained.

(a) Whether an antibody obtained binds to TLR7 on cell surfaces or not
(b) Whether levels of inflammatory cytokines secreted from immunocytes are suppressed or not when an antibody obtained is contacted with the immunocytes while the immunocytes are stimulated with a TLR7 ligand
(c) Whether growth of B cells is suppressed or not when an antibody obtained is contacted with the B cells while the B cells are stimulated with a TLR7 ligand
(d) Whether the pathological condition of an inflammatory disease model animal such as a mouse is improved or not through administration of an antibody obtained to the animal

The anti-TLR7 antibody according to the present invention is useful for diagnosis and treatment of diseases. Examples of diseases for which the anti-TLR7 antibody according to the present invention is particularly useful include various autoimmune diseases. Examples of autoimmune diseases include rheumatoid arthritis (RA), systemic lupus erythematosus (SLE), scleroderma, polymyositis (PM), Sjogren's syndrome, ANCA-associated vasculitis, Behcet's disease, Kawasaki's disease, mixed cryoglobulinemia, multiple sclerosis, Guillain-Barre syndrome, myasthenia, type I diabetes mellitus, Basedow's disease, Hashimoto's disease, Addison's disease, IPEX, APS type-II, autoimmune myocarditis, interstitial pneumonia, bronchial asthma, autoimmune hepatitis, primary biliary cirrhosis, Crohn's disease, ulcerative colitis, psoriasis, atopic dermatitis, hemolytic anemia, autoimmune thyroiditis, and idiopathic juvenile arthritis of polyarthritis type. Especially, the anti-TLR7 antibody according to the present invention is considered to be useful for SLE and psoriasis, for which it has been reported that TLR7 response is involved in the onset mechanism.

The anti-TLR7 antibody according to the present invention in one embodiment contains a pharmaceutically acceptable carrier or additive. Examples of carriers and additives include, but are not limited to, water, saline, phosphate buffer, dextrose, pharmaceutically acceptable organic solvents such as glycerol and ethanol, collagen, polyvinyl alcohol, polyvinyl pyrrolidone, carboxyvinyl polymer, carboxymethyl cellulose sodium, sodium polyacrylate, sodium alginate, water-soluble dextran, carboxymethyl starch sodium, pectin, methyl cellulose, ethyl cellulose, xanthan gum, gum arabic, casein, agar, polyethylene glycol, diglycerin, glycerin, propylene glycol, petrolatum, paraffin, stearyl alcohol, stearic acid, human serum albumin, mannitol, sorbitol, lactose, and surfactants.

Analysis Using Anti-Human TLR7 Antibody

In one aspect, the anti-human TLR7 antibody according to the present invention is used for detecting human TLR7. Specifically, the present invention relates to a method for detecting human TLR7, comprising recognizing or detecting human TLR7 in a sample by using an anti-human TLR7 antibody. The field of application of the present invention covers a wide range of applications including clinical practice, pharmaceuticals, and foods, and the present invention can be used, needless to say, for analysis on autoimmune diseases in which TLR7 is involved, and analysis results from the present invention can be used as indexes in determining administration of an antibody for TLR7.

The present invention relates to, in one aspect, a method for examining a disease in which human TLR7 is involved, comprising recognizing or detecting human TLR7 by using an anti-human TLR7 antibody.

For the recognition or detection, an antibody against human TLR7 or a functional fragment thereof can be used. Detection of the molecule of interest with an antibody or a functional fragment thereof can be performed by using a method well-known to those skilled in the art, and human TLR7 is detected by means of enzyme immunoassay, radioimmunoassay, fluoroimmunoassay, luminescence immunoassay, flow cytometry, a Western blotting method, an immunoprecipitation method, immunoturbidimetry, immunodiffusion, and so on.

Here, a functional fragment of an antibody refers to an antibody fragment with the function of the antibody maintained, and, for example, a functional fragment of an antibody against human TLR7 refers to an antibody fragment with the function of recognizing human TLR7 maintained. For such an antibody fragment, for example, a fragment of an antibody such as scFv and a domain antibody is preferably used.

In a preferred embodiment of the present invention, human TLR7 is detected by enzyme immunoassay, immunoprecipitation, or flow cytometry.

Currently, enzyme immunoassay (EIA), as typified by an ELISA (Enzyme-Linked Immuno Sorbent Assay) method, is used as a method for detecting and/or quantifying a target substance by utilizing antibody-antigen affinity in a wide range of fields, and has become one of techniques essential for various diagnoses and a variety of biological examinations. Immunosensors, biosensors as an application of enzyme immunoassay, have been developed, and widely used.

The principle of measurement by the ELISA method is such that an antigen or antibody as a target substance is reacted with an antibody or antigen linked to a labeling enzyme, and the target substance is detected and/or quantified from the enzyme activity of the labeling enzyme. That is, the ELISA method achieves high-sensitivity detection of a target substance by using an enzyme to amplify a signal acquired through molecular recognition by an antibody.

In the present invention, any labeling enzyme commonly used for the ELISA method and the like can be used without limitation. Preferred examples of labeling enzymes can include peroxidase, alkaline phosphatase, glucose oxidase, and galactosidase, but other enzymes can be also used. In the ELISA method and the like, a method of detecting a reaction product generated through reaction catalyzed by a labeling enzyme from absorption, fluorescence, or luminescence is commonly used, and, in the case of immunosensors, a method of electrochemically detecting enzymatic reaction due to a labeling enzyme is the mainstream. Known as another method for detecting enzymatic reaction is, for example, a method of generating a nonsoluble product through reaction of alkaline phosphatase or the like as a labeling enzyme and detecting the precipitate by using a piezoelectric element such as a quartz crystal unit.

While a variety of methods are widely known as enzyme immunoassay, the present invention is applicable to any known enzyme immunoassay. The present invention is also applicable to immunosensors as an application of enzyme immunoassay.

In the present invention, enzyme immunoassay refers to a method of detecting and/or quantifying a target substance by utilizing enzymatic reaction and immunoreaction. More specifically, enzyme immunoassay is such a method that antigen-antibody reaction is performed by using an antigen or antibody labeled with an enzyme and the antigen-antibody reaction is detected by measuring enzyme activity, and thereby a target substance is detected and/or quantified. Generally known as enzyme immunoassay are competitive methods and noncompetitive methods (e.g., a sandwich method). In competitive methods, an antigen or antibody is labeled with a labeling enzyme, and the labeled antigen is allowed to compete with a free antigen in a sample and undergo antigen-antibody reaction with the corresponding antibody or antigen. Thereafter, a substrate is added to amplify the signal of the antigen-antibody reaction through enzymatic reaction, and detection and/or quantification is performed. In the sandwich method common as a noncompetitive method, on the other hand, a non-labeled antibody (capturing antibody, first antibody) is bound to a target substance (antigen) in a sample, and a labeled antibody (detecting antibody, second antibody), which has been enzyme-labeled, is then bound to the complex of the antigen and the capturing antibody. Thereafter, a substrate is added to amplify the signal of the antigen-antibody reaction through enzymatic reaction, and detection and/or quantification of the target substance is performed. In particular, the sandwich method has high specificity because detection is performed with two different antibodies. In addition, a double layer method and so on are known as enzyme immunoassay, and the present invention is applicable to these measurement methods. In the sandwich method and the like, the capturing antibody and the detecting antibody typically differ in their epitopes.

In linking a labeling enzyme and an antibody, the linking method is not limited, and a chemical procedure or a gene engineering procedure may be used. A labeling enzyme may be directly linked to an antibody, or indirectly bound to an antibody.

In the case that a labeling enzyme is linked to an antibody with a chemical procedure, they can be directly linked together, or linked together via a linker or a spacer. Alternatively, a labeling enzyme and an antibody can be linked together by utilizing specific binding by digoxigenin, avidin, biotin, or the like. In the case that a labeling enzyme is linked to an antibody with a gene engineering procedure, an enzyme-labeled antibody can be produced as a fusion protein (chimeric protein), for example, by using a fusion protein method.

Specifically, known methods including a method using a divalent crosslinking agent can be used for linking of a labeling enzyme to an antibody, but the linking method is not limited thereto. Accordingly, for example, an amino group, a carboxyl group, a hydroxy group, a thiol group, an imidazole group, or a phenyl group can be used. In the case that amino groups are linked together, for example, an isocyanate method, a glutaraldehyde method, a difluorobenzene method, or a benzoquinone method can be used. In the case that an amino group and a carboxyl group are bonded together, a method of converting a carboxyl group into a succinylimide ester can be applied, and in addition a carbodiimide method, a Woodward reagent method, and a periodic acid oxidation method to crosslink an amino group and a glycan (Nakane method) can be applied. Further, in the case that a thiol group is used, for example, a carboxyl group in one side is converted into a succinylimide ester, with which cysteine is reacted to introduce a thiol group, which can be bonded to another side with a divalent crosslinking agent reactive with thiol groups. Furthermore, a diazotization method, an alkylation method, and so on are applicable as a method using a phenyl group.

In the case that an antibody has no appropriate functional group to bind to an enzyme, an amino group, a carboxyl group, a thiol group, or the like may be introduced to it. In this case, a spacer may be allowed to mediate the introduction to facilitate binding to the enzyme.

The linking ratio between an antibody and a labeling enzyme is not limited to 1:1, and any ratio can be employed. For example, a plurality of labeling enzymes can be linked to a molecular recognition element by using the glutaraldehyde method or periodic acid method (J. Histochemistry and Cytochemistry 22, 1084, 1974).

Ina preferred embodiment of the present invention, TLR7 as an antigen can be analyzed through the immunoprecipitation method. The immunoprecipitation method, which utilizes the phenomenon that a soluble antigen and antibody specifically react together to become insolubilized, can detect/separate/purify an antigen under relatively mild conditions. For example, a substrate and an antibody are insolubilized as a large structure by crosslinking many molecules of them. The antibody is typically bound to a carrier such as Sepharose beads, and a method using magnetic beads is also performed in many cases. Immunoprecipitation is easier to perform for polyclonal antibodies than for monoclonal antibodies.

In the immunoprecipitation method, an antibody without specificity to a sample (if used, together with a carrier) may be mixed to remove components that are nonspecifically adsorbed through centrifugation. In the case that magnetic beads are used, separation with a magnet is performed instead of centrifugation.

In one embodiment of the present invention, an antibody or TLR7 as an antigen can be immobilized to a base material. In the case that the present invention is applied to the ELISA method, for example, the antibody of the present invention is immobilized to a base material. For the base material to fix an antibody or an antigen thereto, any known material can be used in the present invention. Accordingly, inorganic polymer compounds such as porous glass, silica gel, and hydroxyapatite, and metals such as gold, silver, and platinum are applicable as a base material. Additional examples include synthetic polymers such as ethylene-vinyl acetate copolymer, polyvinyl chloride, polyurethane, polyethylene, polystyrene, nylon, polyester, and polycarbonate; natural polymers such as starch, gluten, chitin, cellulose, and natural rubber; and derivatives of them. Agarose derivatives having a hydrophobic group, nitrocellulose, and derivatives of them can be also examples of a base material to be used in the present invention. In particular, a material according to a measurement method to be used can be selected for the base material in the present invention, and the shape of the base material is not limited, and the base material can be in the shape of any of a microplate, beads, a film, a sheet, a tube, a fiber, a stick, and so on, according to the usage.

In the case that an antigen or an antibody is immobilized to a base material in the present invention, the immobilizing method is not limited and any known method can be used. For example, physical adsorption, entrapping immobilization, or immobilization by chemical bonding reaction can be used. Examples of physical adsorption include adsorption of protein onto hydrophobic resin surfaces. Entrapping immobilization is such a method that immobilization is achieved by allowing a support such as gel and polymer to entrap a substance to be immobilized. The immobilization method based on chemical bonding is a method of chemically bonding a functional group introduced to the surface of a support to a functional group of a substance to be immobilized.

Examples of the chemical immobilization method include immobilization of an antibody or an antigen to a base material by using a silane coupling agent, a plasma-polymerized film, or an acid anhydride. In the case that an antibody or an antigen is immobilized onto a base material by covalent bonding via an acid anhydride, for example, an antibody and so on may be immobilized via an acid anhydride group present on the surface of a base material, or immobilized via an acid anhydride group introduced in advance to another reactive functional group present on the surface of a base material, such as a carboxyl group, a formyl group, an amino group, an azide group, an isocyanate group, a chloroformyl group, and an epoxy group. In the case that neither an acid anhydride group nor a reactive functional group is present on the surface of a polymer material, an acid anhydride group may be directly introduced to the material surface for immobilization, or a reactive functional group and then an acid anhydride group may be introduced for immobilization. An acid anhydride group can be introduced through reaction with styrene-maleic anhydride copolymer, ethylene-maleic anhydride copolymer, methyl vinyl ether-maleic anhydride copolymer, or the like, or alternatively through graft polymerization of maleic anhydride onto polyurethane by using a 7-ray or an electron beam.

In a method for introducing a reactive functional group to the surface of a polymer material, for example, in the case that a carboxyl group is introduced to ethylene-vinyl acetate copolymer, introduction is achieved by saponifying ethylene-vinyl acetate copolymer and then carboxymethylating the resultant. A carboxyl group can be derivatized into an azide group via a hydrazyl group. Moreover, a carboxyl group can be converted even into a chloroformyl group through chlorination with thionyl chloride, acetyl chloride, or the like.

To introduce an amino group to ethylene-vinyl acetate copolymer, it is suitable to subject saponified ethylene-vinyl acetate copolymer to aminoacetalization. An epoxy group can be introduced through reaction with epichlorohydrin, diethylene glycol diglycidyl ether, or the like. An isocyanate group can be introduced through reaction with hexamethylene diisocyanate, tolylene diisocyanate, or the like. A formyl group can be introduced through reaction with glutaraldehyde, dialdehyde starch, or the like.

In the present invention, a variety of samples can be analyzed. It is preferred in the present invention to analyze samples from living bodies such as blood (whole blood, plasma, serum), lymph fluid, saliva, urine, and diseased tissue sections, and fetal cells present in amniotic fluid and a part of in vitro dividing ova can be used as a specimen. It is needless to say that samples to be analyzed with the present invention may be subjected to treatment such as pretreatment, as necessary. For example, these specimens can be directly used, or concentrated as a precipitate, as necessary, through centrifugation or the like and then subjected to cell disruption treatment, for example, with enzyme treatment, heat treatment, surfactant treatment, ultrasonication, or a combination of them in advance before use. In a preferred embodiment, the detection method of the present invention detects soluble TLR7 in a sample derived from human body fluid. The body fluid is, for example, blood (whole blood, plasma, serum), lymph fluid, tissue fluid, coelomic fluid, digestive juice, saliva, or urine, and more preferably plasma or serum.

In the detection method of the present invention in one embodiment, human TLR7 in a sample is detected by using a plurality of anti-human TLR7 antibodies, as in the sandwich ELISA method. In the case that human TLR7 is detected with the sandwich ELISA method, for example, a first anti-human TLR7 antibody is immobilized to a solid phase carrier, to which a sample derived from human body fluid is added and reacted, and then a second anti-human TLR7 antibody that recognizes a different epitope and is labeled with an enzyme is further added and reacted. After washing followed by reaction with the enzyme substrate for development of color, the human TLR7 level can be determined through measurement of the absorbance. For the first antibody and the second antibody, anti-human TLR7 antibodies according to the present invention for different epitopes can be arbitrarily combined. Examples of such combinations include a combination of an antibody that binds within the region from position 276 to position 313 of the amino acid sequence of human TLR7 represented by SEQ ID NO: 17 (e.g., rE3) and another anti-human TLR7 antibody (e.g., LTM3).

Examination of diseases associated with human TLR7 can be performed by detecting and quantifying human TLR7 present in a sample in the above-described manner. Accordingly, one aspect of the present invention is an agent for examination. For example, if the level of human TLR7 present in a sample is lower than a predetermined threshold, the case is determined as being suspicious to have a disease associated with human TLR7. A cutoff value can be set through statistical analysis using an ROC curve with measurements for samples derived from healthy individuals and disease-affected patients.

The effect of a medicine targeting human TLR7 in a human can be evaluated by detecting and quantifying human TLR7 present in a sample. Accordingly, the anti-human TLR7 antibody according to the present invention is useful as a companion diagnostic agent for a medicine targeting human TLR7. In this case, if the level of human TLR7 in a sample derived from a test subject is lower than a predetermined threshold, the medicine can be predicted to have therapeutic effect in the test subject. Setting of the predetermined threshold is as described above. Here, examples of medicines targeting human TLR7 include medicines for various autoimmune diseases. Examples of autoimmune diseases include rheumatoid arthritis (RA), systemic lupus erythematosus (SLE), scleroderma, polymyositis (PM), Sjogren's syndrome, ANCA-associated vasculitis, Behcet's disease, Kawasaki's disease, mixed cryoglobulinemia, multiple sclerosis, Guillain-Barre syndrome, myasthenia, type I diabetes mellitus, Basedow's disease, Hashimoto's disease, Addison's disease, IPEX, APS type-II, autoimmune myocarditis, interstitial pneumonia, bronchial asthma, autoimmune hepatitis, primary biliary cirrhosis, Crohn's disease, ulcerative colitis, psoriasis, atopic dermatitis, hemolytic anemia, autoimmune thyroiditis, and idiopathic juvenile arthritis of polyarthritis type. Especially, the anti-human TLR7 antibody according to the present invention is considered to be useful in evaluating the effect of a medicine in a human for SLE and psoriasis, for which it has been reported that an anti-TLR7 antibody is involved in the onset mechanism.

The present invention provides, in one aspect, a measurement kit for human TLR7 as a target substance. The measurement kit of the present invention for the target substance includes, in a certain embodiment, an anti-human TLR7 antibody. The measurement kit of the present invention may further include a base material to which the antibody is immobilized, a standard solution, a sensitizer, a buffer, an instruction manual, a package, and so on.

### EXAMPLES

The present invention will be described in more detail with reference to specific experiment examples; however, the present invention is not limited to the experiment examples below. Herein, concentrations and so on are based on weight, and numerical ranges shown are intended to include the endpoints, unless specifically stated.

### Experiment 1: Acquisition of Anti-hTLR7 Antibody

(1) Vector Construction and Establishment of hTLR7-Expressing Cells

Into a retroviral vector (pMXs) in which six Flag-His tags had been added to the C-terminal side of the gene, a hTLR7 gene (full-length, a gene encoding the amino acid sequence represented by SEQ ID NO: 17) was incorporated by using an In Fusion enzyme (Takara Bio Inc.). This retroviral vector was transfected into a packaging cell line (Plat-E, derived from HEK293 cells) by using a transfection reagent (Fugene 6, F. Hoffmann-La Roche Ltd.). After 24 hours, the culture supernatant was collected for use as a virus suspension. This virus suspension was mixed well with a liposomal transfection reagent (DOTAP, F. Hoffmann-La Roche Ltd.) and the mixture was added to the target cells, and centrifugal treatment was performed at 2000 rpm for 1 hour. The retroviral vector and the packaging cell line were given by Professor KITAMURA Toshio at The Institute of Medical Science, The University of Tokyo.

### (2) Immunization

(LTM3) To each TLR9-deficient mouse (background: BALB/c), an antigen (Ba/F3 cell line forcedly expressing hTLR7-Flag-Hisx6/hUnc93B1-HAx2) mixed with an adjuvant (Titer MAX Gold, CYT) was administered to the foot sole, to the root of the tail, and intraperitoneally every week, three times in total. At fourth administration, the antigen suspended in phosphate-buffered saline (PBS) was intraperitoneally administered. The spleen was isolated from each mouse 5 days after the date of the final immunization, and used for producing hybridomas.

(rE3) To each wild-type rat (background: Wister), an antigen (Ba/F3 cell line forcedly expressing hTLR7-Flag-Hisx6/hUnc93B1-HAx2) mixed with an adjuvant (Titer MAX Gold, CYT) was administered to the foot sole, to the root of the tail, and intraperitoneally every week, three times in total. At fourth administration, the antigen suspended in phosphate-buffered saline (PBS) was intraperitoneally administered. The spleen was isolated from each rat 5 days after the date of the final immunization, and used for producing hybridomas.

### (3) Production of Hybridomas

Splenocytes after the immunization were mixed with an Sp2/O mouse myeloma cell line, and cell fusion was performed by using an HVJ-E cell fusion kit (ISHIHARA SANGYO KAISHA, LTD.). The next day of the cell fusion operation, culture with HAT-containing culture solution (culture solution containing hypoxanthine, aminopterin, and thymidine) was initiated to select hybridomas.

### (4) Screening Test for Hybridomas

With a microscope, culture supernatant was collected from each hybridoma forming a colony and used for screening. Cell membrane permeabilization was performed by using 0.1% saponin solution, and the Ba/F3 cell line forcedly expressing hTLR7-Flag-Hisx6/hUnc93B1-HAx2 and a Ba/F3 cell line without expression were stained with the culture supernatant, and analyzed through flow cytometry to select hybridomas producing an anti-hTLR7 antibody.

### (5) Determination of Gene Sequence for Antibody Variable Region

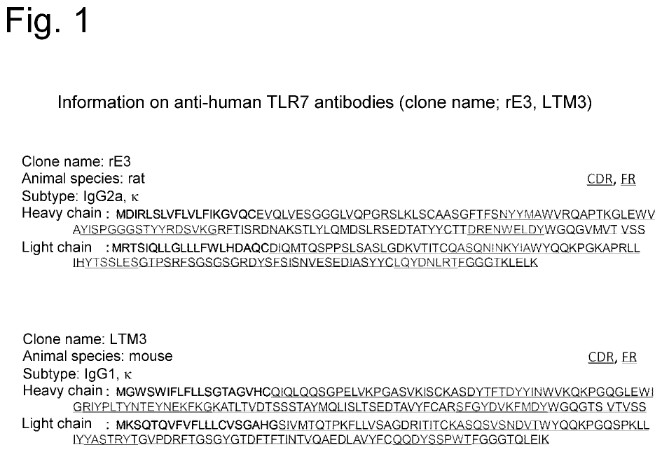

Each hybridoma for an antibody was subjected to gene sequence analysis (performed by GenScript Biotech Corporation). FIG. 1 shows the results.

### Experiment 2: Specificity Evaluation for Monoclonal Antibodies Through Flow Cytometry

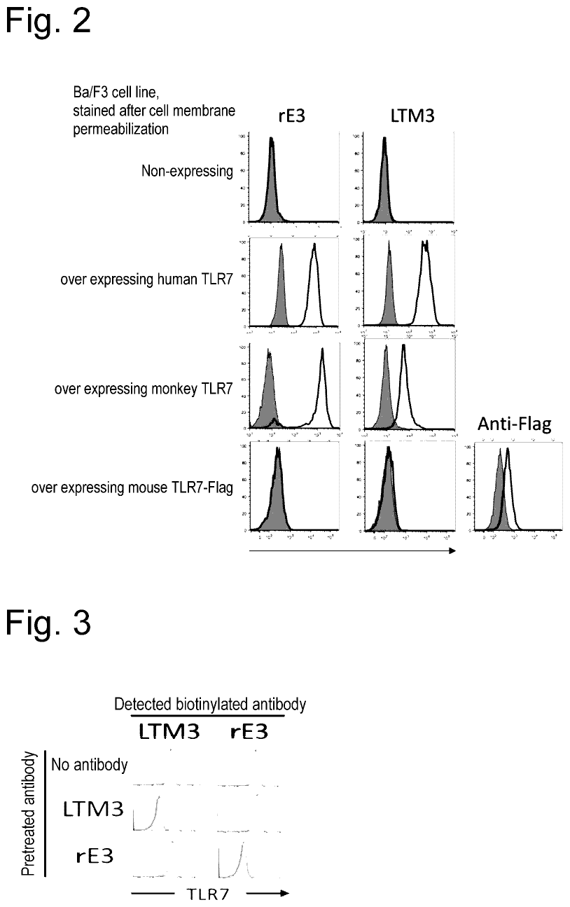

A Ba/F3 cell line forcedly expressing hTLR7-Flag-Hisx6/hUnc93B1-HAx2, monkey TLR7, or mouse TLR7 was subjected to cell membrane permeabilization using 0.1% saponin solution, and stained with rE3 or LTM3. For evaluation of binding of each antibody, analysis was performed through flow cytometry and histograms were compared with those for staining of non-expressing cells. The results showed that rE3 and LTM3 exhibited cross-activity not only to human TLR7 but also to monkey TLR7, but did not show cross-activity to mouse TLR7 (FIG. 2).

### Experiment 3: Establishment of ELISA System

Because rE3 and LTM3 differ in their epitopes (FIG. 3), sandwich ELISA (Enzyme-Linked Immuno Sorbent Assay) using these monoclonal antibodies was performed. Specifically, the LTM3 antibody and a biotinylated rE3 antibody (rE3-bio) were used as a capturing antibody and a detecting antibody, respectively, and an antigen was detected by using a direct detection method.

(1) Preparation of Plate

LTM3 diluted with PBS (10 μg/ml) was seeded in a 96-well plate, and left to stand at 4° C. overnight. After washing three times with PBS (containing 0.05% by weight of Tween), blocking was performed with 4-fold-diluted blocking reagent (Blocking One, NACALAI TESQUE, INC.).

(2) Preparation of Specimens

Samples collected from healthy individuals were used as specimens (serum: seven specimens, plasma: nine specimens). To a solution obtained by 10-fold-diluting blocking reagent (Blocking One, NACALAI TESQUE, INC.) (hereinafter, referred to as dilution solution), a surfactant (Triton-X100) was added to reach a final concentration of 0.1%. By using this dilution solution, 5 of each specimen was 10-fold-diluted. Monkey TLR7 protein (extracellular domain, macaca TLR7) with a known concentration was subjected to serial dilution with the dilution solution from 8 ng/ml to 0.03125 ng/ml at 2-fold intervals to prepare specimens for preparing a standard curve. The monkey TLR7 protein was donated by Dr. SHIMIZU Toshiyuki at Faculty of Pharmaceutical Science, The University of Tokyo.

(3) Detection of hTLR7

A plate was washed three times, and 1 μg/ml of a biotinylated rE3 antibody was added thereto. The plate was left to stand at room temperature for 2 hours. After washing three times, an avidin-HRP complex was added, and the plate was left to stand at room temperature for 30 minutes.

After washing three times, an HRP substrate (horseradish peroxidase substrate) was added, and reacted for 15 minutes. Subsequently, 1 N sulfuric acid was added to quench the reaction.

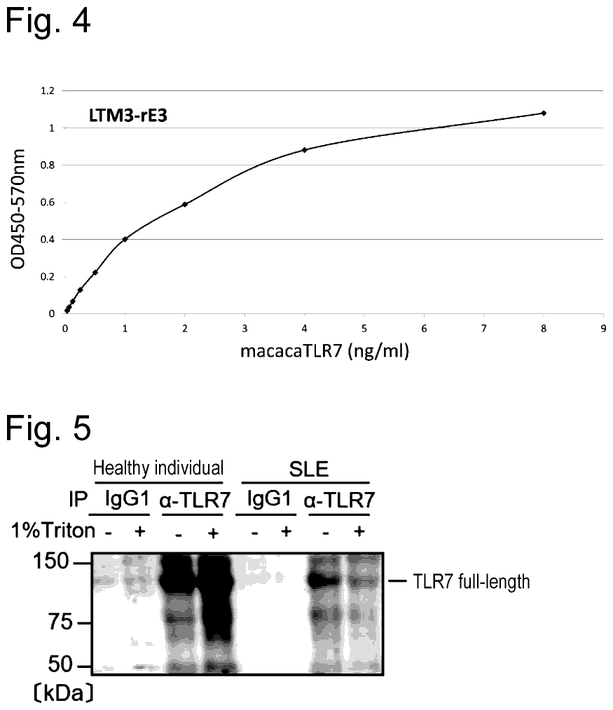

Absorbance at a wavelength of 450 nm was then measured, and absorbance at a wavelength of 570 nm was appropriately removed. The standard curve for monkey TLR7 is shown in FIG. 4.

TABLE 1

| TLR7 concentration | OD |
|---|---|
| (ng/ml) | (450 nm-570 nm) |
| 0 | 0.0025 |
| 0.03125 | 0.0175 |
| 0.0625 | 0.036 |
| 0.125 | 0.067 |
| 0.25 | 0.129 |
| 0.5 | 0.223 |
| 1.0 | 0.402 |
| 2.0 | 0.5895 |
| 4.0 | 0.882 |
| 8.0 | 1.081 |

(4) Quantification Using Standard Curve

Soluble TLR7 was measured for a sample obtained from plasma of a healthy individual or an SLE (systemic lupus erythematosus) patient to quantify soluble TLR7 on the basis of the standard curve, and the results were as follows.
healthy individual: 3246 pg/ml
SLE patient: 273 pg/ml

Thus, soluble TLR7 was actually quantified successfully through sandwich ELISA using a plurality of antibodies differing in their antigenic determinants.

### Experiment 4: Detection of Soluble TLR7 Through Immunoprecipitation and Western Blotting Method

Treated with 500 μl of a surfactant (Triton-X100, final concentration: 1%) was 500 μl of human serum or plasma. Subsequently, immunoprecipitation was performed by using rE3 supported on a carrier (NHS-activated Sepharose 4 Fast Flow, GE Healthcare).

These specimens were then separated by molecular weight through SDS-PAGE, and hTLR7 was detected through Western blotting. For detection of hTLR7, the following antibodies were used.
Primary antibody: Rabbit Anti-TLR7 monoclonal antibody (Cell Signaling Technology, Inc. (CST))
Secondary antibody: Anti-Rabbit IgG (H+L) antibody-HRP (Thermo Fisher Scientific)

A chromogenic substrate was added, and the molecular weight of each band detected was evaluated. FIG. 5 shows the results, where a band of about 130 kDa was detected, which was determined to correspond to full-length hTLR7.

### Experiment 5: Detection of Soluble TLR7 Through ELISA

By using the ELISA system established in Experiment 3, serum or plasma derived from human peripheral blood was analyzed to examine the relation to diseases. Specifically, TLR7 was quantified for serum or plasma derived from peripheral blood collected from a systemic lupus erythematosus (SLE), dermatomyositis (DM), polymyositis (PM), systemic scleroderma (SSc), or mixed connective tissue disease (MCTD) patient, or a healthy individual (Healthy).

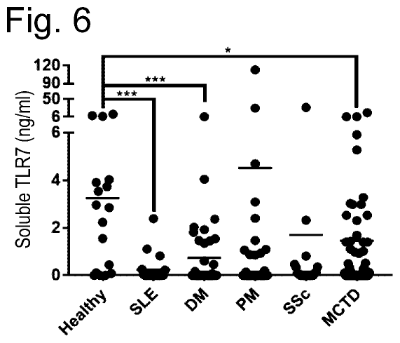

FIG. 6 shows the analysis results, where clear significant difference was found for the samples derived from systemic lupus erythematosus (SLE) and dermatomyositis (DM) patients to the samples derived from healthy individuals (***). In addition, significant difference was found for the samples derived from polymyositis (PM), systemic scleroderma (SSc), and mixed connective tissue disease (MCTD) patients to the samples derived from healthy individuals (*). For example, an ROC curve was drawn with the result for the group of patients with systemic lupus erythematosus, and compared with the result for the samples derived from healthy individuals to determine a concentration with the highest Youden index, and the concentration was found to be 1327 pg/ml. For example, samples can be analyzed with this numerical value set as a threshold.

These results demonstrate that soluble TLR7 is useful as a marker indicating pathological condition of autoimmune diseases, and detection of TLR7 with the present invention before administration of an antibody against TLR7 is usefully applicable to diagnosis and treatment of autoimmune diseases.

### INDUSTRIAL APPLICABILITY

Anti-human TLR7 antibodies were established, and an assay system capable of detecting soluble TLR7 was constructed. Analysis of human serum gave a result that soluble TLR7 was detected in the serum. Moreover, it was revealed that significant difference in the level of soluble TLR7 is present between serum derived from healthy individuals and that derived from autoimmune disease patients. Results obtained by using the detection system are considered to be useful as an index indicating the state of pathological condition in such diseases that TLR7 is involved in pathological condition.

1. A method for analyzing a human-derived body fluid sample, comprising:
detecting human TLR7 in a sample from a test subject by using an anti-human TLR7 antibody; and
comparing a detection result on the sample from the test subject with a detection result on a sample derived from a healthy individual.

2. The method according to claim 1, wherein the human TLR7 is detected through enzyme immunoassay and/or immunoprecipitation.

3. The method according to claim 1, wherein the human TLR7 in each of the samples is detected by using a plurality of anti-human TLR7 antibodies.

4. The method according to claim 1, wherein the sample is a sample derived from human body fluid.

5. The method according to claim 1, wherein the sample comprises human plasma and/or serum.

6. The method according to claim 1, wherein the test subject is a patient having an autoimmune disease or an individual suspected to have an autoimmune disease.

7. An agent for examining an autoimmune disease involving activation of TLR7, comprising an anti-human TLR7 antibody.

8. The agent according to claim 7, for analyzing a human-derived body fluid sample.

9. The agent according to claim 7, wherein the autoimmune disease is SLE.
